(19) United States
(12) Patent Application Publication
(10) Pub. No.: US 2024/0344031 A1
(43) Pub. Date: Oct. 17, 2024

(54) MESENCHYMAL STEM CELL LINE

(71) Applicant: UNIVERSITÄT BASEL, Basel (CH)

(72) Inventors: Sebastien PIGEOT, Huningue (FR); Ivan MARTIN, Oberwil (CH); Paul BOURGINE, Lund (SE)

(73) Assignee: UNIVERSITÄT BASEL, Basel (CH)

(21) Appl. No.: 18/682,918

(22) PCT Filed: Aug. 2, 2022
(86) PCT No.: PCT/EP2022/071731

§ 371 Date: Feb. 11, 2024

(30) Foreign Application Priority Data
Aug. 11, 2021 (EP) 21190889.2

(51) Int. Cl. C12N 5/0775; C07K 14/51; C12N 5/077; C12N 9/12; C12N 9/64

(52) U.S. Cl. CPC C12N 5/0662; C07K 14/51; C12N 5/0655; C12N 9/1276; C12N 9/6472; C12N 2506/1346; C12N 2510/00; C12N 2533/90; C12Y 207/07049; C12Y 304/22036

(57) ABSTRACT

A mesenchymal stem/stromal (MSC) cell line, in particular a human mesenchymal stem cell (hMSC), capable of chondrogenic differentiation when cultured in a chondrogenic medium, comprising a first transgene comprising a first nucleic acid sequence encoding for a preferably mammalian immortalizing enzyme under control of a first promoter sequence operable in said mesenchymal cell and a second transgene comprising a second nucleic acid sequence encoding a preferably mammalian bone morphogenic protein under control of a second promoter sequence operable in said mesenchymal cell.

### TECHNICAL FIELD

The present invention relates to a mesenchymal stem/stromal (MSC) cell line, in particular a mesenchymal stem cell of human origin (hMSC), capable of chondrogenic differentiation when cultured in a chondrogenic medium, as well as to a method to generate a decellularized hypertrophic cartilage graft material using such cells, and their use in generating a decellularized hypertrophic cartilage graft material.

### BACKGROUND AND PRIOR ART

Bone repair is among the most potent regenerative processes in the human body, typically healing without scar tissue. In some critical cases, however, spontaneous bone healing is not sufficient and surgical intervention using graft substitutes is required.

The current gold standard for bone grafting consists in autologous bone transplant. Nevertheless, there are significant drawbacks, such as limited material availability, increased risks of infection, logistics, and morbidity at the donor site.

Alternative strategies not relying on transplants rely on materials with limited biological functionality (e.g., xeno-, allo-graft, synthetic scaffolds) or on osteoinductive growth factors delivered at supra-physiological doses to force bone formation, raising safety concerns due to the side-effect associated with supra-physiological doses.

Therefore, there is a crucial need for novel bone repair strategies that combine the benefits of autologous bone transplants with the ease of availability of the alternatives without, or less of, their drawbacks.

A recently proposed approach for bone repair was reported in “Orthotopic Bone Formation by Streamlined Engineering and Devitalization of Human Hypertrophic Cartilage” Sébastien Pigeot, Paul Emile Bourgine, Jaquiery Claude, Celeste Scotti, Adam Papadimitropoulos, Atanas Todorov, Christian Epple, Giuseppe M. Peretti and Ivan Martin, Int. J. Mol. Sci. 2020, 21, 7233, where it was reported that the an engineered but cell-free material, when tested at an orthotopic site, was demonstrated to lead to a superior formation of de novo bone tissue as compared to a clinical standard-of-care allografts obtained from processing bone tissue of human donors.

The engineered and cell-free material was hypertrophic cartilage that was provided via the 3D perfusion culturing of transduced human bone marrow mesenchymal cells (hBM-MSCs) after inducing chondrogenic differentiation on a type I collagen substrate and subsequently decellularizing. The hBM-MSCs included transgenes that allowed to trigger apoptosis in the cells via an inducible modified caspase 9 (iCas9).

WO2013/186264 A1 discloses methods to provide human bone marrow-derived mesenchymal stem cells having a chemically inducible apoptosis system and being immortalized. It further discloses methods to generate devitalized tissue grafts via 3D perfusion culturing of transduced human bone marrow mesenchymal stem cells on a porous ceramic substrate after inducing differentiation. It is noted that while osteogenic and adipogenic differentiation was successful to some extent no chondrogenic differentiation could be shown even though the cells were cultured in chondrogenic medium.

“Combination of cell immortalization and apoptosis induction to engineer decellularized matrices as bone graft materials” Bourgine, Paul, 2013, Doctoral Thesis, University of Basel, Faculty of Science, discloses in Chapter 2, methods to provide human bone marrow-derived mesenchymal stem cells having a chemically inducible apoptosis system and being immortalized. Osteogenic and adipogenic differentiation were tested in 2D culture while chondrogenic differentiation in macromass aggregates. It is noted that while osteogenic and adipogenic differentiation was successful to some extent no chondrogenic differentiation could be shown even though the cells were cultured in chondrogenic medium. Bone formation capacity of these cells was assessed by direct in vivo implantation mixing porous ceramic granules with the cells using fibrin. No frank bone formation was achieved.

«Osteoinductivity of engineered cartilaginous templates devitalized by inducible apoptosis» Bourgine et al. PNAS, 2014 discloses chondrogenic and hypertrophic cartilage tissue formation from primary hMSC engineered with the apoptotic cassette and subsequent osteoinductivity of the devitalized tissue in vivo.

### SUMMARY OF THE INVENTION

The present invention provides a cell line that can be differentiated into chondrogenic lineage and which reliably exhibits osteoinductive ECM production, which ECM consists of superior hypertrophic cartilage graft material that can be stored and used easily.

It is thus an object of the present invention to provide a mesenchymal stem/stromal (MSC) cell line, in particular a human mesenchymal stem cell (hMSC), capable of chondrogenic differentiation when cultured in a chondrogenic medium, comprising
a. a first transgene comprising a first nucleic acid sequence encoding for an a preferably mammalian immortalizing enzyme under control of a first promoter sequence operable in said mesenchymal cell
b. a second transgene comprising a second nucleic acid sequence encoding a preferably mammalian bone morphogenic protein under control of a second promoter sequence operable in said mesenchymal cell,
c. optionally not comprising a third transgene encoding for a third nucleic acid sequence encoding a protein capable of triggering apoptosis in said mesenchymal stem cell when said cell is exposed to a triggering agent.

It was found that the constitutive overexpression of a bone morphogenic protein, in particular human bone morphogenic protein 2, restored the chondrogenic differentiation potential to clonal cells, which were selected for their capacity and further expanded. In our experience the clonal selection is critical. Regarding the donor dependency, we believe it could potentially restore chondrogenic differentiation capacities in all donors but it was never checked.

It is further an object of the present invention to provide a method to generate a devitalised hypertrophic cartilage graft material comprising the steps of
a. seeding a substrate scaffold with mesenchymal stem/stromal (MSC) cells of the cell line as claimed in any of the preceding claims,
b. culturing the mesenchymal stem/stromal (MSC) cells in a chondrogenic medium, preferably essentially free of glycerophosphate, until an extracellular matrix (ECM) having at least 0.4 μg GAG/μg DNA and/or 50 pg of BMP2/μg of protein is formed on the substrate scaffold, to form a hypertrophic cartilage graft material,
c. devitalizing the hypertrophic cartilage graft material to obtain a devitalized hypertrophic cartilage graft material.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows photographs of hypertrophic cartilage cultured in vitro for 3 weeks, where glycosaminoglycan (GAG, revealed by Safranin-O staining), collagen type II as well as collagen X are visualized by staining. The hypertrophic cartilage comprising mesenchymal stem/stromal (MSC) cell line according to the present invention is identified by “MB” whereas a cell line of immortalized mesenchymal cell line without BMP-2 overexpression is identified by “M”.

FIG. 2 shows measurements of the glycosaminoglycan (GAG) content, when measured via the Blyscan and normalized to total DNA content measured via the Cyquant assay, in hypertrophic cartilage cultured in vitro for 3 weeks in chondrogenic medium. The mesenchymal stem/stromal (MSC) cell line “MB” according to the present invention yielded hypertrophic cartilage having 4.08 μgGAG/μgDNA and whereas immortalized mesenchymal cell line without BMP-2 overexpression identified by “M” led only to 0.8 μgGAG/μgDNA.

FIG. 3 shows measurements of bone morphogenic protein 2 (BMP2) content in hypertrophic cartilage hypertrophic cartilage tissue cultured in vitro for 3 weeks, in chondrogenic medium. The hypertrophic cartilage comprising mesenchymal stem/stromal (MSC) cell line according to the present invention is identified by “MB” whereas a cell line of immortalized mesenchymal cell line without BMP-2 overexpression is identified by “M”. The BMP2 content in the tissue was quantified by ELISA and normalized to total protein content within the tissue using BCA assay.

FIG. 4 shows photographs of hypertrophic cartilage at different bone maturities 0, 1, 2, 3, and 4, a higher number reflecting higher bone maturity. Bone maturity is quantified for each by computing a “bone score”, which corresponds to the sum of the extent of bone (B) and bone marrow (BM) features as identified by specific staining. The respective percentage areas of B and BM are manually determined over the total surface area of the tissue section using FIJI image analysis software. For each sample, three sections were assessed at various depths. A score from 0 (no B or BM) to 4 (over 90% of B+BM area) was given to each section and the average of the scores was retained as the ossicle maturity score for the whole sample. A minimum of three samples were analyzed for each experimental group.

FIG. 5 shows the evolution of hypertrophic cartilage obtained with a mesenchymal stem/stromal (MSC) cell line according to the present invention (identified by “MB”) and a cell line of immortalized mesenchymal cell line without BMP-2 overexpression (identified by “M”), when seeded on a bovine collagen type I scaffold and cultured for 3 weeks either in chondrogenic (identified as “-C”) or osteogenic (identified as “-O”) medium, after subcutaneous implantation in vivo in nude mice.

### DESCRIPTION OF PREFERRED EMBODIMENTS

It is an object of the present invention to provide a mesenchymal stem/stromal (MSC) cell line, in particular a human mesenchymal stem cell (hMSC), capable of chondrogenic differentiation when cultured in a chondrogenic medium, comprising
a. a first transgene comprising a first nucleic acid sequence encoding for an a preferably mammalian immortalizing enzyme under control of a first promoter sequence operable in said mesenchymal cell
b. a second transgene comprising a second nucleic acid sequence encoding a preferably mammalian bone morphogenic protein under control of a second promoter sequence operable in said mesenchymal cell,
c. optionally not comprising a third transgene encoding for a third nucleic acid sequence encoding a protein capable of triggering apoptosis in said mesenchymal stem cell when said cell is exposed to a triggering agent.

Human bone marrow-derived Mesenchymal Stem/Stromal Cells (hMSCs) are defined as a cellular fraction positive for CD73, CD90, CD105, and negative for hematopoietic markers, while being able to stably differentiate in vitro into osteoblasts, adipocytes and chondrocytes [Pittenger et al., Science 1999; 284:143-147].

In a preferred embodiment of the mesenchymal stem/stromal (MSC) cell line according to the present invention, the human mesenchymal stem cell of the invention is characterized in that
the cell is positive for CD29, CD44, CD73, CD90, CD146 and/or CD105; and/or
the cell is negative for hematopoietic markers such as CD34 and/or CD45.

It is understood that the term “a cell line positive for a surface marker”, such as for example one or more CD antigens mentioned above, means that at least 90%, and more preferably at least 92%, of the cell line exhibits said marker when sorted, for example via a FACS cell sorter.

It is understood that the term “a cell line negative for a surface marker”, such as for example one or more CD antigens mentioned above, means that at least less than 10%, and more preferably at least less than 5%, of the cell line exhibits said marker when sorted, for example via a FACS cell sorter.

In the context of the present invention, a chondrogenic medium corresponds to a medium that, when human mesenchymal stem/stromal cells are cultured in said medium, induces the differentiation of the cultured human mesenchymal stem/stromal cells into chondrocytes, i.e. chondrogenic differentiation. Chondrogenic differentiation of the cultured human mesenchymal stem/stromal cells may be ascertained via multiple markers known in the field. One such marker is the increased expression of glycosaminoglycan (GAG) or collagen type II, which may be detected via known methods such as Blyscan colorimetric assay or immunohistological methods.

An exemplary chondrogenic medium suitable for inducing the differentiation of the cultured human mesenchymal stem/stromal cells into chondrocytes consists of DMEM containing 4.5 mg/ml D-Glucose, 0.1 mM nonessential amino acids, 1 mM sodium pyruvate, 100 mM HEPES buffer, 100 U/ml penicillin, 100 mg/ml streptomycin, and 0.29 mg/ml L-glutamine supplemented with 0.1 mM ascorbic acid 2-phosphate, 10 ng/ml TGFb1 and 10⁷ M dexamethasone, as was previously described [Jakob et al., Journal of Cellular Biochemistry 81: 368±377 (2001].

The applicants found that for the cells of the mesenchymal stem/stromal (MSC) cell line according to the present invention to differentiate into chondrocytes, culture in a chondrogenic medium, for about 2 to 3 weeks, is sufficient. It is noted that after 2 or 3 weeks in culture in a chondrogenic medium, the cells of the mesenchymal stem/stromal (MSC) cell line according to the present invention further differentiate in hypertrophic cartilage.

The mesenchymal stem/stromal (MSC) cell line according to the present invention comprises a first transgene comprising a first nucleic acid sequence encoding for an a preferably mammalian immortalizing enzyme under control of a first promoter sequence operable in said mesenchymal cell.

In preferred embodiment of the mesenchymal stem/stromal (MSC) cell line according to the present invention, the first transgene comprising a first nucleic acid sequence encoding for a preferably mammalian immortalizing enzyme encodes for the gene of a human telomerase catalytic subunit (hTERT) [Jun et al., Cell Physiol Biochem. 2004; 14 (4-6): 261-8]. The inclusion of hTERT in the hMSCs cell line allows a significant extension of the hMSCs life-span (>300 PD) while preserving some of the properties of the primary hMSCs.

In preferred embodiment of the mesenchymal stem/stromal (MSC) cell line according to the present invention, the human telomerase is telomerase reverse transcriptase as defined in GenBank BAC1 1010.

In preferred embodiment of the mesenchymal stem/stromal (MSC) cell line according to the present invention, the first promoter sequence operable in said mesenchymal cell is a promoter leading to constitutive expression of the first transgene comprising first nucleic acid sequence encoding for an immortalizing enzyme, in particular is a cytomegalovirus (CMV) promoter taken from the Genbank cloning vector KJ697753.1.

In preferred embodiment of the mesenchymal stem/stromal (MSC) cell line according to the present invention, the first nucleic acid sequence further comprises an expressed transgene facilitating the selection of cells expressing said expressed transgene, such as for example a fluorescent gene like enhanced green fluorescent protein (EGFP) or tandem dimer Tomato (tdTomato) or a surface marker like delta cluster of differentiation 19 (ΔCD19) or delta nerve growth factor receptor (ΔNGFR).

The mesenchymal stem/stromal (MSC) cell line according to the present invention is a cell line of immortalized cells from human mesenchyme origin preferably stably and constantly expressing BMP2. It has been found that the cells of the cell line retain the differentiation features of hMSC such as osteogenic, adipogenic and particularly frank chondrogenic differentiation in 2D and 3D culture.

In preferred embodiment of the mesenchymal stem/stromal (MSC) cell line according to the present invention, the second transgene comprising a second nucleic acid sequence encoding a preferably mammalian bone morphogenic protein under control of a second promoter sequence operable in said mesenchymal cell encodes for the gene for human bone morphogenic protein 2 (BMP-2).

In preferred embodiment of the mesenchymal stem/stromal (MSC) cell line according to the present invention, the second promoter sequence operable in said mesenchymal cell is a promoter leading to constitutive expression of the second transgene comprising the second nucleic acid sequence encoding for a bone morphogenic protein, in particular is a human phosphoglycerate kinase (hPGK) or spleen focus forming virus (SFFV) promoter respectively taken from the pLVX-Puro Vector (CLontech) and the Genbank cloning vector KJ697753.1.

In preferred embodiment of the mesenchymal stem/stromal (MSC) cell line according to the present invention, the second nucleic acid sequence further comprises an expressed transgene facilitating the selection of cells expressing said expressed transgene, such as for example a fluorescent gene like EGFP, tdTomato, or a surface marker like CD19 or ΔNGFR.

In preferred embodiment of the mesenchymal stem/stromal (MSC) cell line according to the present invention, the cell line does not comprise a third transgene encoding for a third nucleic acid sequence encoding a protein capable of triggering apoptosis in said mesenchymal stem cell when said cell is exposed to a triggering agent.

In preferred embodiment of the mesenchymal stem/stromal (MSC) cell line according to the present invention the protein capable of triggering apoptosis is a caspase, particularly caspase 9 (Uniprot ID P5521 1).

In preferred embodiment of the mesenchymal stem/stromal (MSC) cell line according to the present invention, the mesenchymal stem/stromal (MSC) cell line is the cell line hTERT_BT_clon1 and deposited on 21 Jul. 2021 with the Culture Collection of Switzerland (CCOS) under accession number CCOS 1983 having the following particulars:
Name: Prof. Ivan Martin
Depositor: University Hospital Basel
   ZLF/Lab 405
   Hebelstarsse 20
   4031 Basel
   Switzerland

The cell line deposited on 21 Jul. 2021 with the Culture Collection of Switzerland (CCOS) under accession number CCOS 1983 comprised a transgene comprising a first nucleic acid sequence encoding for a catalytic subunit of a human telomerase hTERT, as well as a transgene comprising a first nucleic acid sequence encoding human bone morphogenic protein 2 under the control of a SFFV promoter, and no transgene encoding for a nucleic acid sequence encoding a protein capable of triggering apoptosis in said mesenchymal stem cell when said cell is exposed to a triggering agent.

In preferred embodiment of the mesenchymal stem/stromal (MSC) cell line according to the present invention, the mesenchymal stem/stromal (MSC) cell line is the cell line MB_10-12 and deposited on 21 Jul. 2021 with the Culture Collection of Switzerland (CCOS) under accession number CCOS 1984 having the following particulars:
Name: Prof. Ivan Martin
Depositor: University Hospital Basel
   ZLF/Lab 405
   Hebelstarsse 20
   4031 Basel
   Switzerland

The cell line deposited on 21 Jul. 2021 with the Culture Collection of Switzerland (CCOS) under accession number CCOS 1984 comprised a transgene comprising a first nucleic acid sequence encoding for a catalytic subunit of a human telomerase hTERT, as well as a transgene comprising a first nucleic acid sequence encoding human bone morphogenic protein 2 under the control of a hPGK promoter, and no transgene encoding for a nucleic acid sequence encoding a protein capable of triggering apoptosis in said mesenchymal stem cell when said cell is exposed to a triggering agent.

In preferred embodiment of the mesenchymal stem/stromal (MSC) cell line according to the present invention, the mesenchymal stem/stromal (MSC) cell line is the cell line designated MB_0 and deposited on 21 Jul. 2021 with the Culture Collection of Switzerland (CCOS) under accession number CCOS 1985 having the following particulars:
Name: Prof. Ivan Martin
Depositor: University Hospital Basel
   ZLF/Lab 405
   Hebelstarsse 20
   4031 Basel
   Switzerland

The cell line deposited on 21 Jul. 2021 with the Culture Collection of Switzerland (CCOS) under accession number CCOS 1985 comprised a transgene comprising a first nucleic acid sequence encoding for a catalytic subunit of a human telomerase hTERT, as well as a transgene comprising a first nucleic acid sequence encoding human bone morphogenic protein 2 under the control of a CMV promoter, and a transgene encoding for a nucleic acid sequence encoding a protein capable of triggering apoptosis in said mesenchymal stem cell when said cell is exposed to a triggering agent, i.e. caspase 9 (Uniprot ID P5521 1).

It is further an object of the present invention to provide a method to generate a devitalised hypertrophic cartilage graft material comprising the steps of
a. seeding a substrate scaffold with mesenchymal stem/stromal (MSC) cells of the cell line as claimed in any of the preceding claims,
b. culturing the mesenchymal stem/stromal (MSC) cells in a chondrogenic medium, preferably essentially free of glycerophosphate, until a an extracellular matrix (ECM) having at least 0.4 μg GAG/μg DNA and/or 50 pg of BMP2/μg of protein is formed on the substrate scaffold, to form a hypertrophic cartilage graft material,
c. devitalizing the hypertrophic cartilage graft material to obtain a devitalized hypertrophic cartilage graft material

In the context of the present invention, the term “devitalisation” is the process used to isolate the extracellular matrix (ECM) of a tissue from its inhabiting cells, leaving an ECM scaffold of the original tissue without living cells.

In the context of the present invention, the term “decellularization” is the process used to isolate the extracellular matrix (ECM) of a tissue from its inhabiting cells, leaving an ECM scaffold of the original tissue essentially without cell components.

In the method to generate a devitalised hypertrophic cartilage graft material according to the present invention, the substrate scaffold is seeded with mesenchymal stem/stromal (MSC) cells of the cell line as described above, and preferably is seeded with an amount of cells in the range of 10 to 200 M cells per ml, preferably of 15 to 120 M cells per ml of culture medium. In an exemplary embodiment, the amount of cells is about 60M cells per ml. It is noted that seeding can for example be achieved by immersing the substrate scaffold in the a cell culture comprising cells in the above-mentioned amounts. Alternatively, the substrate scaffold may be seeded with mesenchymal stem/stromal (MSC) cells of the cell line as described above by mixing the constituents of the substrate scaffold with mesenchymal stem/stromal (MSC) cells of the cell line as described above to form a mixture, and 3D printing said mixture such as to obtain the desired form for the seeded substrate scaffold, which is the cultured in a chondrogenic medium.

In the method to generate a devitalised hypertrophic cartilage graft material according to the present invention, the seeded mesenchymal stem/stromal (MSC) cells are cultured in a chondrogenic medium, preferably essentially free of glycerophosphate, until an extracellular matrix (ECM) having at least 0.4 μg GAG/μg DNA and/or 50 pg of BMP2/μg of protein is formed on the substrate scaffold, to form a hypertrophic cartilage graft material.

In a preferred embodiment of the method to generate a devitalised hypertrophic cartilage graft material according to the present invention, the seeded mesenchymal stem/stromal (MSC) cells are cultured in a chondrogenic medium until the extracellular matrix (ECM) having at least 0.5 μg GAG/μg DNA, more preferably having at least 0.8 μg GAG/μg DNA, even more preferably having at least 1 μg GAG/μg DNA and most preferably having at least 1.5 μg GAG/μg DNA, is formed on the substrate scaffold.

The applicants have found that, for a sufficient amount of extracellular matrix to be deposited on the scaffold, culturing the chondrocytes in chondrogenic medium for about 2 weeks, preferably for 3 weeks, is in general sufficient.

In a preferred embodiment of the method to generate a devitalised hypertrophic cartilage graft material according to the present invention, the chondrogenic medium is essentially free of glycerophosphate. Glycerophosphate is a well-known constituent of hypertrophic medium, which was believed to be necessary for the formation of hypertrophic cartilage graft material. It was however found that neither the use of a separate hypertrohic medium or the inclusion of glycerophosphate was necessary for the formation of a hypertrophic cartilage graft material and that the chondrogenic medium in and of itself, in combination with the cell line of the present invention, was sufficient to drive the formation of hypertrophic cartilage graft material.

In a preferred embodiment of the method to generate a devitalised hypertrophic cartilage graft material according to the present invention, the substrate scaffold comprises or consists of an organic material, in particular a protein such as collagen. In particular, collagen I can be used, and is commercially available in required amounts and purity.

In a preferred embodiment of the method to generate a devitalised hypertrophic cartilage graft material according to the present invention, step a., step b. and/or step c. are carried out in a perfusion bioreactor system.

In a preferred embodiment of the method to generate a devitalised hypertrophic cartilage graft material according to the present invention, the substrate scaffold is a 2D or 3D substrate scaffold. A 2D scaffold may for example be a sheet or slab of substrate scaffold, whereas 3D scaffolds may be spheres or granules, either individual or packed into a bed, cubes, and or more complex shapes. It is understood that the substrate scaffold is a substrate scaffold that can be obtained via 3D printing as described above or by casting a substrate scaffold constituent into a mold.

The devitalised hypertrophic cartilage graft material according to the present invention is osteoinductive and can be used for treating bone defects. Osteoinduction is the process by which an implanted material recruits host (immature) cells to differentiate and form de novo bone through chemical/biological cues. In contrast, osteoconduction is the process by which bone is formed on an implanted material from existing adjacent bone tissue, serving as a guiding scaffold for the new bone to grow on.

### Experimental Data

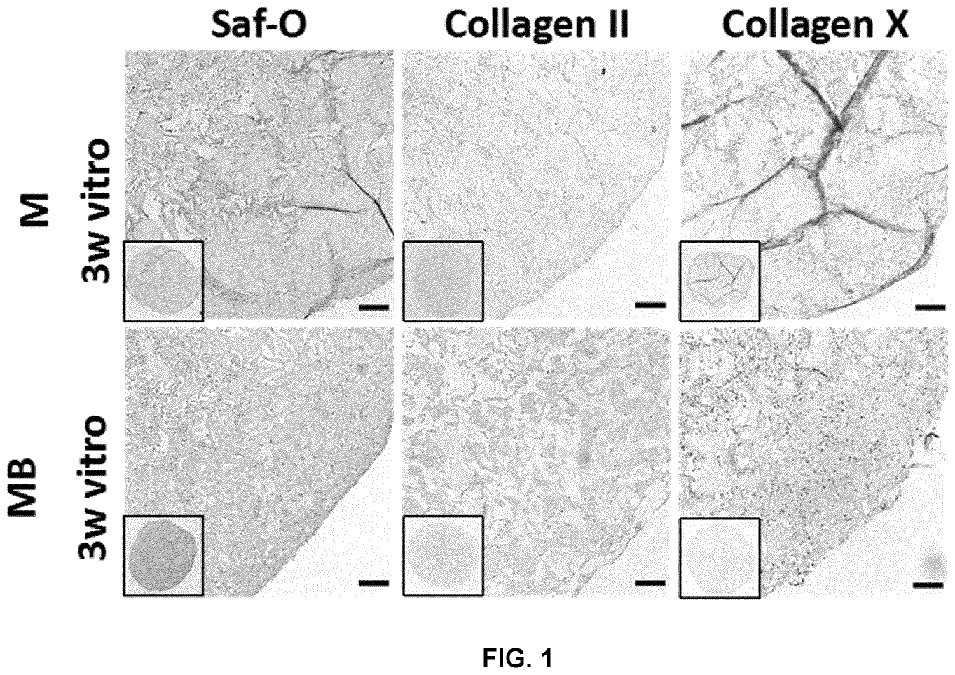

As can be seen from FIG. 1, each of the visualized cartilage components, i.e. GAG (revealed in red by Safranin-O staining), collagen II and collagen X are more evenly distributed, and more intensely colored in the photographs depicting the cartilage obtained with the cell line according to the present invention, which overexpresses BMP2 (Label “MB”) than the cartilage obtained with immortalized MSC (Label “M”) not overexpressing BMP2. This means that the cell line according to the present invention, in contrast to the comparative cell line, allows for a restoration of chondrogenic differentiation when cultured in a chondrogenic medium.

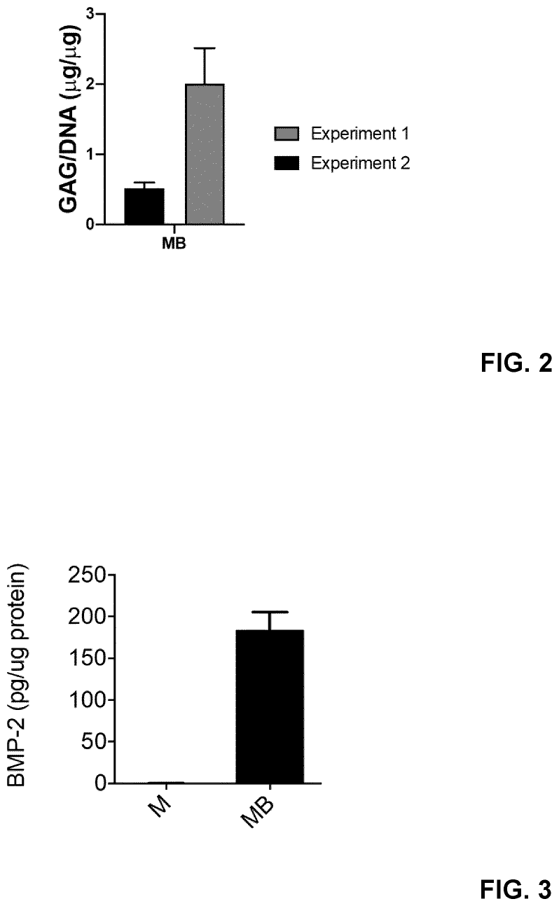

As can be seen from FIG. 2, measurements of the glycosaminoglycan (GAG) content, when measured via the Blyscan assay and normalized to total DNA content measured via the Cyquant assay, in hypertrophic cartilage cultured in vitro for 3 weeks in chondrogenic medium show that the mesenchymal stem/stromal (MSC) cell line according to the present invention yielded hypertrophic cartilage having at least 0.4 μg GAG/μg DNA (Experiment 1) and up to or in excess of 2 μg GAG/μg DNA (Experiment 2).

As can be seen from FIG. 3, when cultured in chondrogenic medium, the cell line according to the present invention (Label “MB”), when compared to a cell line of immortalized mesenchymal cells with (Label “M”) exhibits substantial constitutive expression of BMP2, after culture for 3 weeks in chondrogenic medium. The amount of BMP2 in the tissue was quantified by ELISA and normalized to total protein content within the tissue using BCA assay.

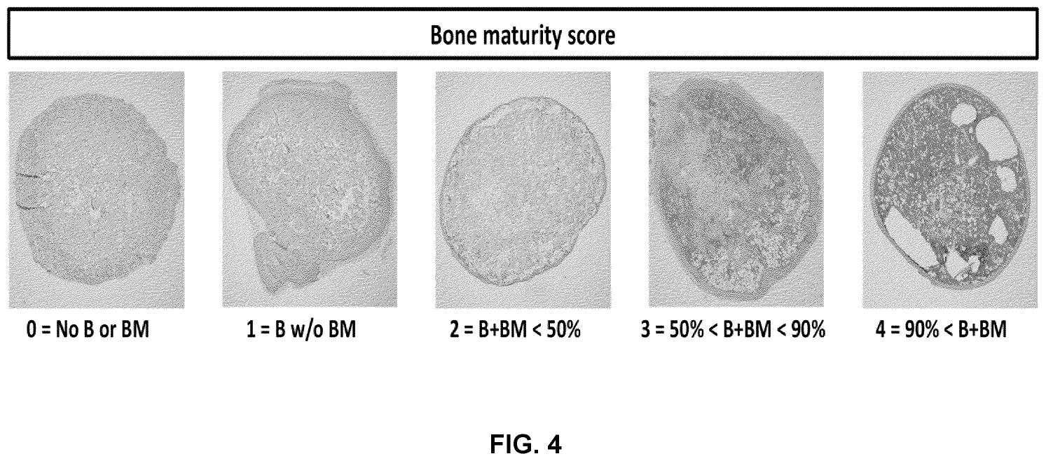

As can be seen from FIG. 4, hypertrophic cartilage can be quantified in terms of bone maturity. Bone maturity is quantified for each sample by computing a “bone score”, which corresponds to the sum of the extent of bone (B) and bone marrow (BM) features as identified by specific staining. The respective percentage areas of B and BM are manually determined over the total surface area of the tissue section of the sample using FIJI image analysis software. For each sample, three sections were made and assessed at various depths. A score from 0 (no B or BM) to 4 (over 90% of B+BM area) was given to each section and the average of the scores was retained as the ossicle maturity score for the whole sample. A minimum of three samples were analyzed for each experimental group.

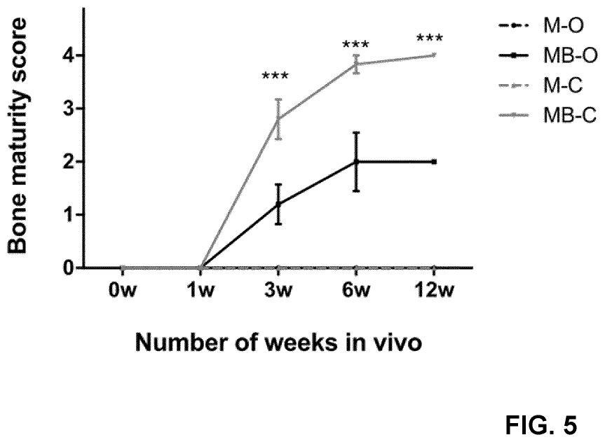

As can be seen from FIG. 5, the in vivo evolution of devitalized hypertrophic cartilage obtained with a mesenchymal stem/stromal (MSC) cell line according to the present invention (identified by “MB”) is superior to hypertrophic cartilage obtained with a cell line of immortalized mesenchymal cell line without BMP-2 overexpression (identified by “M”). After seeding on a bovine collagen type I scaffold and culturing for 3 weeks in chondrogenic (identified as “-C”) medium, the hypertrophic cartilage obtained with a mesenchymal stem/stromal (MSC) cell line according to the present invention (identified by “MB”) reaches near full bone maturity 4 (indicative of osteoinductivity), at six weeks after subcutaneous implantation. In contrast, when the same cells are seeded on bovine collagen type I scaffold and cultured for 3 weeks in osteogenic (identified as “-O”) medium, the obtained osteogenic tissue stagnates at a lower bone maturity of about 2 at six weeks after subcutaneous implantation. As a comparative, tissues obtained with a cell line of immortalized mesenchymal cell line without BMP-2 overexpression (identified by “M”), shows no significant evolution of bone maturity beyond a bone maturity of 0 in either osteogenic (identified as “-O”) medium or chondrogenic (identified as “-C”) medium over 12 weeks. Thus, this demonstrates BMP-2 overexpression enables the maturation of hypertrophic cartilage towards higher bone maturity (osteoinductivity) in vivo, when subcutaneously implanted, i.e. without requiring the proximity of bone tissue. Further, if mesenchymal stem/stromal (MSC) cell line according to the present invention are cultured in chondrogenic medium prior to implantation, the hypertrophic cartilage are obtained quickly and fully mature into bone tissue.

### Material and Methods

### Cell Culture

Human bone marrow derived mesenchymal stromal cells (hBM-MSCs) were isolated from human bone marrow aspirates obtained from routine orthopedic surgical procedures involving exposure of the iliac crest, after ethical approval (Ethikkommission beider Basel, Ref.78/07) and informed donor consent.

Briefly, marrow aspirates (20 ml volume) were harvested from healthy donors using a bone marrow biopsy needle inserted through the cortical bone and immediately transferred into plastic tubes containing 15,000 IU heparin. After diluting the marrow aspirate with phosphate buffered saline (PBS) at a ratio of 1:4, nucleated cells were counted and seeded at a density of 3.106 cells/cm² in complete medium (CM, α-minimum essential Medium (αMEM) with 10% fetal bovine serum, 1% HEPES (1 M), 1% Sodium pyruvate (100 mM) and 1% of Penicillin-Streptomycin-Glutamine (100×) solution (all from Gibco)) supplemented with 5 ng/ml of fibroblast growth factor-2 (FGF-2, R&D Systems) and cultured in a humidified 37° C./5% CO2 incubator. hBM-MSCs were selected based on adhesion and proliferation on the plastic substrate one week after seeding. Cells were then detached using trypsin-EDTA 0.05% (Gibco), counted and seeded at a density of 3′000 cells/cm² for expansion. MSOD (M) and MSOD-BMP2 (MB) cells were cultured in the same condition as hBM-MSCs with a seeding density of 6′000 cells/cm².

### Lentivirus Production and Cellular Transduction

The lentivirus expressing BMP2/tdTomato was generated using 3rd generation lentiviral vector system. Human codon optimized BMP2 was designed and synthetized by GeneArt using human BMP2 RefSeq NM_001200 as reference. tdTomado coding sequence was obtained from Addgene (Plasmid #32904). BMP2 and tdTomato CDS were fused by PCR with a T2A self-cleaving peptide inserted between both coding sequences. The resulting fusion product was subcloned into modified pLVX lentiviral expression vector (Clontech) in which PGK promoter and puromycin resistance gene were removed.

For lentivirus production, Lenti-X 293T cells (Clontech) were transfected with lentiviral expression vector and 3rd generation packaging plasmids prMDLg/pREE, pRSV-Rec and pMD2.G (Addgene #12251, #12253 and #12259, respectively) using Lipofectamine2000 (Lifetech). 72 hours after transfection, the supernatant containing lentiviral particles was collected. Lentivirus preparation was concentrated using PEG-it Virus precipitation kit (System Biosciences) and the concentrated virus was re-suspended in sterile phosphate buffered saline (PBS) and stored at −80° C. until use. Lentiviral titer was assessed by ELISA using Quick Titer Lentivirus titer kit (Cell Biolabs).

BM196-hTERT at passage 15 were plated at a density of 100′000 cells/well in a 6-well plate the day preceding the transduction. Cells were transduced overnight by incubation with BMP-2-tdTomato lentivirus vector at a multiplicity of infection of 10, followed by fresh medium replacement. Cells stably expressing hTERT-GFP and BMP-2-tdTomato were single-cell sorted in 6×96 well plates using a FACS-Melody cell sorter (Becton Dickinson, Basel, Switzerland). 39 surviving and proliferating clones could be further expanded and assessed for their capacity to form cartilage extracellular matrice enriched in BMP-2.

### Phenotypic Analysis

The immunophenotypic analysis of hBM-MSCs, MSOD and MSOD-B lines was performed using LSR II FORTESSA SORP (BD Biosciences) cell analyzer. Cells were harvested by regular trypsinization step and labeled at 4° C. for 20 min with following fluorochrome-conjugated antibodies diluted in PBS supplemented with 2% FBS and 0.5 mM EDTA: human anti-CD34 (BioLegend cat #343512), human anti-CD45 (BD BIOSCIENCES cat #560973), human anti-CD29 (BioLegend, cat #303014), human anti-CD44 (BD BIOSCIENCES cat #559942), human anti-CD73 (BD BIOSCIENCES cat #561014), human anti-CD90 (BD BIOSCIENCES cat #559869), human anti-CD146 (BioLegend, cat #342003). Positive expression was defined based on superior fluorescence intensity than the respective unstained and isotype controls.

### Generation of In Vitro 3D Cartilage or Osteogenic Tissues

Tissues were cultured statically in standard 12 well culture plates coated with 2% agarose to prevent cells from attaching and proliferating at the bottom. Cells (MSOD, MSOD-B) were seeded at a density of 2 million cells in 35 μl on type I collagen sponges previously punched to a diameter of 6 mm (Avitene™ Ultrafoam™, BD). After a 1 h incubation time at 37° C. to allow cell attachment, constructs were primed toward chondrogenic or osteogenic differentiation for 3 weeks to achieve cartilage or osteogenic tissue formation. Chondrogenic medium consisted of DMEM supplemented with penicillin-streptomycin-glutamine (Gibco), HEPES (Gibco), sodium pyruvate (Gibco), ITS-A (Insulin, Transferrin, Selenium) (Gibco), Human Serum Albumin 0.12% (CSL Behring), 0.1 mM ascorbic acid (A5960, Sigma), 10−7 M dexamethasone (D4902, Sigma) and 10 ng/ml TGF-β3 (Novartis).

### Tissue Lyophilization

Tissues were transferred to screw cap eppendorf tubes and snap frozen in liquid nitrogen (LN2) prior to lyophilization. Lyophilizer (Martin Christ, Alpha 2-4 LSCplus) was set at a temperature and pressure respectively lower than −40° C. and 0.05 mbar overnight with the tubes slightly unscrewed to allow for tissue water sublimation. Upon retrieval, tissues were either stored at 4° C., implanted in vivo after rehydration or analyzed.

### Glycosaminoglycans (GAG) Measurements

GAG content in culture supernatants was assessed using the Barbosa method 28. Briefly, 250 μl of collected supernatant was incubated with 1 ml of DMMB solution (16 mg/l dimethylmethylene blue, 6 mM sodium formate, 200 mM GuHCL, all from Sigma Aldrich, pH 3.0) on a shaker at room temperature for 30 minutes. After centrifugation, precipitated DMMB-GAG complexes were dissolved in decomplexion solution (4 M GuHCL, 50 mM Na-Acetate, 10% Propan-1-ol, all from Sigma Aldrich, pH 6.8) at 60° C. for 15 minutes. Absorption was measured at 656 nm and corresponding GAG concentrations were calculated using a standard curve prepared with purified bovine chondroitin sulfate (Sigma Aldrich).

For measurement of GAG content in cartilage tissue, samples were preliminary digested overnight at 56° C. in 1 ml of proteinase K solution (Sigma Aldrich, P2308), and 100 μl of the resulting digested solution was used for the DMMB-GAG precipitation.

### BMP-2 Quantification

BMP-2 protein content within supernatant and tissues was assessed using the human BMP-2 DuoSet ELISA (R&D Systems) according to the manufacturer's instructions.

### Histological Analysis

Samples used for histology were embedded in paraffin and sections of 5 μm thickness were prepared using a microtome (Microm, HM430, Thermo Scientific). Safranin-O stainings were performed as previously described (Scotti et al. PNAS, 110, 3997-4002, 2013).

### In Vivo Implantation

All animal studies were approved by the corresponding ethical authorities in Sweden or Switzerland. In Sweden, mouse experiments were approved by the Swedish Board of Agriculture (animal ethical permit 15485-18). In Switzerland, experiments were approved by the Swiss Federal Veterinary Office (permit 1797 for the mice). Analgesia was provided 1 h prior to surgery by subcutaneous injection of Buprenorphin (0.1 mg/kg body weight). Anesthesia with isofluran (2.5%) was maintained on demand with oxygen as a carrier (0.6 l/min). Prior to surgery the fur was shaved if necessary and disinfected with 70% ethanol. For subcutaneous implantation in mice, 6 to 10 weeks old female CD-1 nude mice were used. Two midline incisions (c.a. 5 mm) of the dorsal skin were performed using scissors under sterile conditions and up to four tissues were implanted per animal.

### LIST OF REFERENCE SIGNS

none

1. A mesenchymal stem/stromal (MSC) cell line, in particular a human mesenchymal stem cell (hMSC), capable of chondrogenic differentiation when cultured in a chondrogenic medium, comprising
a. a first transgene comprising a first nucleic acid sequence encoding for a preferably mammalian immortalizing enzyme under control of a first promoter sequence operable in said mesenchymal cell
b. a second transgene comprising a second nucleic acid sequence encoding a preferably mammalian bone morphogenic protein under control of a second promoter sequence operable in said mesenchymal cell,
c. optionally not comprising a third transgene encoding for a third nucleic acid sequence encoding a protein capable of triggering apoptosis in said mesenchymal stem cell when said cell is exposed to a triggering agent.

2. The mesenchymal stem/stromal (MSC) cell line as claimed in claim 1, wherein the first transgene comprising a first nucleic acid sequence encoding for an immortalizing enzyme is a transgene comprising a first nucleic acid sequence encoding for a catalytic subunit of a human telomerase, in particular hTERT.

3. The mesenchymal stem/stromal (MSC) cell line as claimed in claim 1, wherein the first promoter sequence operable in said mesenchymal cell is a promoter leading to constitutive expression of the first transgene comprising first nucleic acid sequence encoding for an immortalizing enzyme, in particular is a CMV promoter and/or wherein the second promoter sequence operable in said mesenchymal cell is a promoter leading to constitutive expression of the second transgene comprising the second nucleic acid sequence encoding for a bone morphogenic protein, in particular is a hPGK promoter.

4. The mesenchymal stem/stromal (MSC) cell line as claimed in claim 1, wherein the mammalian bone morphogenic protein corresponds to mammalian bone morphogenic protein 2 (BMP-2), preferably human bone morphogenic protein 2 (BMP-2).

5. A mesenchymal stem/stromal (MSC) cell line as claimed in claim 1, wherein said a human telomerase is telomerase reverse transcriptase as defined in GenBank BAC1 1010; said protein capable of triggering apoptosis is a caspase, particularly caspase 9 (Uniprot ID P5521 1); and/or said first and/or said second nucleic acid sequence further comprises an expressed transgene facilitating the selection of cells expressing said expressed transgene, such as for example a fluorescent gene like EGFP, tdTomato, or a surface marker like CD19 or ΔNGFR.

6. A method to generate a devitalised hypertrophic cartilage graft material comprising the steps of
a. seeding a substrate scaffold with mesenchymal stem/stromal (MSC) cells of the cell line as claimed in claim 1,
b. culturing the mesenchymal stem/stromal (MSC) cells in a chondrogenic medium, preferably essentially free of glycerophosphate, until a an extracellular matrix (ECM) having at least 0.4 μg GAG/μg DNA and/or 50 pg of BMP2/μg of protein is formed on the substrate scaffold, to form a hypertrophic cartilage graft material,
c. devitalizing the hypertrophic cartilage graft material to obtain a devitalized hypertrophic cartilage graft material.

7. The method as claimed in claim 6, wherein the substrate scaffold comprises an organic material, in particular a protein such as collagen.

8. The method as claimed in claim 6, wherein step a., step b. and/or step c. are carried out in a perfusion bioreactor system.

9. The method as claimed in claim 6, wherein the substrate scaffold is a 2D or 3D substrate scaffold and/or wherein the devitalized hypertrophic cartilage graft material is lyophilized after step c.

10. An osteoinductive devitalized hypertrophic cartilage graft material obtained according to the method as claimed in claim 6.

11. An osteoinductive devitalized hypertrophic cartilage graft material obtained according to the method as claimed in claim 6 for use in the treatment of a bone defect, in particular bone fracture or defects occurring during spinal bone fusion.

12. A mesenchymal stem/stromal (MSC) cell line as claimed in claim 1, capable of chondrogenic differentiation when cultured in a chondrogenic medium, wherein the MSC cell line is represented by the cell line deposited on 21 Jul. 2021 with the Culture Collection of Switzerland (CCOS) under accession number CCOS 1983.

13. A mesenchymal stem/stromal (MSC) cell line as claimed in claim 1, capable of chondrogenic differentiation when cultured in a chondrogenic medium, wherein the MSC cell line is represented by the cell line deposited on 21 Jul. 2021 with the Culture Collection of Switzerland (CCOS) under accession number CCOS 1984.

14. A mesenchymal stem/stromal (MSC) cell line as claimed in claim 1, capable of chondrogenic differentiation when cultured in a chondrogenic medium, wherein the MSC cell line is represented by the cell line deposited on 21 Jul. 2021 with the Culture Collection of Switzerland (CCOS) under accession number CCOS 1985.
